(19) United States
(12) Plant Patent Application Publication
(10) Pub. No.: US 2003/0188364 P1
(43) Pub. Date: Oct. 2, 2003

(54) Impatiens plant named 'Fify Blush Pink'

(76) Inventor: Johanna Bea Jonkers, RK Hoorn (NL)

Correspondence Address: ROTHWELL, FIGG, ERNST & MANBECK, P.C., 1425 K STREET, N.W., SUITE 800, WASHINGTON, DC, 20005, US

(21) Appl. No.: 10/106,190

(22) Filed: Mar. 27, 2002

(51) Int. Cl. A01H 5/00

(52) U.S. Cl. PLT/317

(57) ABSTRACT

A impatiens cultivar particularly distinguished by light pink colored flowers, compact habit and good basal branching.

### GENUS AND SPECIES

Impatiens walleriana

### VARIETY DENOMINATION

‘Fify Blush Pink’

### BACKGROUND OF THE NEW PLANT

The present invention comprises a new and distinct cultivar of impatiens, botanically known as Impatiens walleriana, and hereinafter referred to by the cultivar name ‘Fify Blush Pink’. The new cultivar is asexually reproduced from vegetative cuttings and tissue culture resulting from the cross of the seed/pod parent IMN-1807-6, a proprietary line that is unnamed and unpatented×IMO-2574-1, a proprietary line that is unnamed and unpatented.

‘Fify Blush Pink’ is a product of a planned breeding program intended to create new impatiens plants with light pink colored flowers, compact habit, good basal branching and moderately vigorous growth.

The new cultivar was created in 1997 in Andjik, The Netherlands and has been asexually reproduced repeatedly by vegetative cuttings and tissue culture in Gilroy, Calif. and Andjik, The Netherlands, over a four year period. The plant has also been trialed at Gilroy, Calif., Litchfield, Mich. and Andijk, The Netherlands. The present invention has been found to retain its distinctive characteristics through successive propagations; and this novelty is firmly fixed.

### DESCRIPTION OF PHOTOGRAPH

This new impatiens plant is illustrated by the accompanying photograph which shows blooms, buds, and foliage of the plant in full color, the colors shown being as true as can be reasonably obtained by conventional photographic procedures.

FIG. 1 shows overall plant habit;

FIG. 2 shows the mature inflorescence.

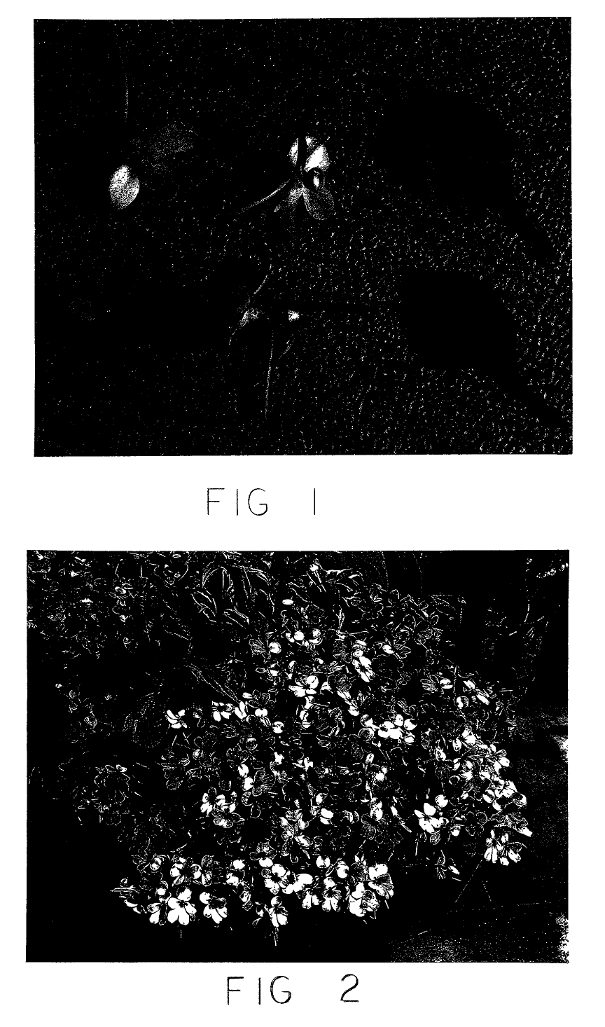

### DESCRIPTION OF THE NEW CULTIVAR

The following detailed descriptions set forth the distinctive characteristics of ‘Fify Blush Pink’. The data which defines these characteristics were collected from asexual reproductions carried out in Gilroy, Calif. The plant history was taken on 21 week old plants grown in one gallon pots, in a poly-covered greenhouse under natural light and color readings were taken in the greenhouse. Color references are primarily to the RHS Colour Chart of The Royal Horticultural Society of London (RHS). Texture description details were observed under a dissecting microscope according to The New Royal Horticultural Society Dictionary of Gardening (1992).

### THE PLANT

Classification:

Botanical.—Impatiens walletiana.

Commercial.—Impatiens.

Form: Compact, small mounded

Growth and branching habit: Compact habit, good basal branching, moderately vigorous, pinching of the terminal apices enhances branching.

Height: From soil level to top of blooms: Approximately 10-12 cm

Width: Approximately 40-43 cm including flowers

Time to produce a finished flowering plant: 6-10 weeks for a 4 inch pot.

Outdoor plant performance: Good shade, intermediate flowering, free-flowering all year, some heat tolerance. Use in hanging baskets, mixed containers, window boxes and mass plantings.

Time to initiate roots: Approximately 10 days in the greenhouse

Time to develop roots: Approximately 21 days in the greenhouse

Root description: Fibrous, fleshy, white

### THE LEAVES

Alternate, whorls

Length: 2.2-2.5 cm

Width: 1.5-2.1 cm

Leaf blade shape: Ovate

Leaf margin: Crenate with bristles between teeth

Apex aspect: Cuspidate

Base aspect: Acuminate

Foliage color: Green color between RHS 137A and 137B.

Texture: Smooth

Venation: Pinnate

Venation color: Green RHS 145D

Petiole length: 1.3-2.3 cm

Petiole diameter: 0.2-0.3 cm

Petiole color: Green RHS 145D

### THE STEM

Length: 19-22 cm

Diameter: 0.4-0.5 cm

Internode length: 2.0 cm

Color: Green RHS 137D

Texture: Smooth

Stem anthocyanin: Slight to moderate in irregular small blotches.

### THE BUD

Shape: Ovate

Diameter: 0.3 cm

Length: 0.4-0.5 cm

Color at tight bud: Pink RHS 68C

### THE FLOWER

Blooming habit: Continuous and freely all through the year.

Inflorescence type: Racemes in leaf axis and in leaf whorls.

Number of flowers per raceme and/or whorl: 10-15 including buds.

Number of flowers per plant: Well over 200

Peduncle length: 1.3-1.5 cm

Peduncle diameter: 0.1 cm

Peduncle color: Yellow-green RHS 144C

Peduncle texture: Smooth

Flower color, front side: Dorsal petal is White RHS 155D with a blushing of red-purple RHS 67C on the front side; a 0.6 cm by 0.1 cm (length×width) of green RHS 145B keel (ridge). Lateral petals are red-purple RHS 66C with slightly darker margins. Flower color, back side: Dorsal petal is white RHS 155D with a blushing of red-purple RHS 66D; margins of RHS 66C and a 0.5 cm by 0.1 cm (length×width) of green RHS 145B keel (ridge).

Eye color and size: Red-purple RHS 63B; 0.1-0.2 cm

Floret form and number of petals: 5 petals; 1 standard or dorsal (free), 2 lateral or lobed pairs (wing petals) deeply lobed.

Floret diameter: Approximately 1.6-1.8 cm

Floret height: 1.8-2.0 cm

Petal size, dorsal: length 0.7-0.8 cm; width 0.8-0.9 cm; shape orbicular; apex shape retuse. Lateral petal: length 1.1-1.2 cm; width 0.6-0.7 cm; shape oblong-obovate; apex shape retuse.

Petal base shape: Fused

Petal margin: Entire

Petal texture: Papillose

Sepals: Three, one larger than the others with a spur.

Large sepal: Color is White RHS 155B with yellow-green RHS 144C apex; length 0.7 cm; width 0.5 cm; shape obovate; apex shape cuspidate; base shape fused; spur color is peach RHS 37D with a length of 2.3-2.5 cm.

Small sepal: Color is yellow-green RHS 144B; length 0.15 cm; width 0.1 cm; shape linear; apex shape acute.

Lastingness of individual blooms: Moderate

Fragrance: None

### THE REPRODUCTIVE ORGANS

Stamens: Five more or less fused by the anthers and covering the ovary; color is red-purple RHS 63B (pollen cap).

Pollen amount and color: Very slight

Pistil: One

Stigma: 5-lobed; red-purple RHS 64A

Fruit seed set: Very minimal.

### DISEASE AND INSECT RESISTANCE

Not observed

### COMPARISON WITH KNOWN CULTIVARS

When the instant plant is compared to ‘Fify White’ (U.S. Plant Pat. No. 11,407) the ‘Fify Blush Pink’ has light pink colored flowers and more flowers per whorl versus white colored flowers and less flowers per whorl for ‘Fify White’. Additionally the leaves of ‘Fify Blush Pink’ are smaller than ‘Fify White’.

I claim:

1. A new and distinct cultivar of impatiens plant as shown and described herein.
